(19) United States
(12) Patent Application Publication
(10) Pub. No.: US 2016/0113859 A1
(43) Pub. Date: Apr. 28, 2016

(54) COSMETIC COMPOSITION COMPRISING A SILICONE OIL AND POLYMETHYLMETHACRYLATE PARTICLES

(71) Applicant: DSM IP ASSETS B.V., Te Heerlen (NL)

(72) Inventors: Achim BROCK, Basel (CH); Christian GSTOETTMAYR, Basel (CH); Aline HUEBER, Basel (CH); Szilvia MESAROS, Basel (CH); Juergen H. VOLLHARDT, Basel (CH)

(21) Appl. No.: 14/890,736

(22) PCT Filed: May 15, 2014
(86) PCT No.: PCT/EP2014/060010

§ 371 Date: Nov. 12, 2015

(30) Foreign Application Priority Data
May 16, 2013 (EP) 13168059.7
May 16, 2013 (EP) 13168079.5

(51) Int. Cl. A61K 8/895; A61Q 19/00; A61K 8/06; A61K 8/02; A61K 8/891

(52) U.S. Cl. CPC A61K 8/895; A61K 8/0241; A61K 8/891; A61K 8/064; A61Q 19/00; A61K 2800/49; A61K 2800/412; A61K 2800/596

(57) ABSTRACT

The present invention relates to a topical composition comprising a silicone oil and a porous cross-linked polymethylmethacrylate bead having a particle size Dᵥ0 of greater 0.3 μm a Dᵥ100 of less than 35 μm, a Dᵥ50 selected in the range of 6-15 μm and an oil absorption capacity selected in the range of 1.2-2.5 cc/g, characterized in that the silicone oil constitutes at least 5 wt.-% of the total oil phase of the topical composition. Furthermore the invention relates to the use of such polymethylmethacrylate beads to boost the silicone feel of a cosmetic composition comprising such silicone oils.

The present invention relates to a topical composition comprising a silicone oil and a porous cross-linked polymethylmethacrylate bead having a particle size Dᵥ0 of greater 0.3 μm, a Dᵥ100 of less than 35 μm, a Dᵥ50 selected in the range of 6-15 μm and an oil absorption capacity selected in the range of 1.2-2.5 cc/g, characterized in that the silicone oil constitutes at least 5 wt.-% of the total oil phase of the topical composition. Furthermore the invention relates to the use of such polymethylmethacrylate beads to boost the silicone feel of a cosmetic composition comprising such silicone oils.

Silicone oils such as cyclomethicone or dimethicone are used in many personal-care products because they spread easily on the hair and skin, and the lubrication they provide isn't greasy or tacky. Furthermore, they provide a silicone like feeling which is highly estimated by the end consumer. However even though silicones have many beneficial effects, there is an ongoing demand from the cosmetic industry to reduce the amount of silicone oils in the cosmetic formulations.

Thus, there is an ongoing need to for topical compositions exhibiting a silicone-like feeling however with as little silicone as possible. Thus, there is a need for substances which boost the perceived silicone effect and thus allow the reduction of the overall used amount of the silicone oil.

Surprisingly it has been found that the addition of relatively low amounts of a specific texturing agent to a cosmetic composition comprising a silicone oil such as cyclomethicone or polysilicone-15 significantly boosts the silicone feeling after application on the skin.

Thus, the present invention relates to a topical composition comprising a silicone oil and a porous cross-linked polymethylmethacrylate bead having a particle size Dᵥ0 of greater 0.3 μm, a Dᵥ100 of less than 35 μm, a Dᵥ50 selected in the range of 6-15 μm and an oil absorption capacity selected in the range of 1.2-2.5 cc/g in an amount selected in the range of 0.5-5 wt.-% based on the total weight of the composition, characterized in that the silicone oil constitutes at least 5 wt.-% of the total oil phase of the topical composition.

Another subject matter of the invention is directed to the use of a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition to improve the silicone feel of a topical composition comprising a silicone oil on the skin, characterized in that the silicone oil constitutes at least 5 wt.-% of the total oil phase of the topical composition.

Furthermore, the invention is directed to a method for improving the silicone feel of a topical composition comprising a silicone oil, said method comprising the step of adding to the topical composition a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition, applying the composition to the skin and appreciating the effect, characterized in that the silicone oil constitutes at least 5 wt.-% of the total oil phase of the topical composition.

According to the invention suitable silicone oils include the volatile cyclic and linear silicones as well as non-volatile silicones such as dialkyl and alkyl aryl siloxanes, as well as cyclic silicones e.g. cyclic and linear polydialkylsiloxanes as well as alkoxylated and/or aminated derivatives thereof, dihydroxypolydimethylsiloxanes and polyphenylalkylsiloxanes.

Cyclic silicones such as cyclopentasiloxane (CAS: 541-02-6), cyclotetrasiloxane (CAS: 556-67-2), cyclohexasiloxane (CAS: 540-97-6), octamethylcyclotetrasiloxane (CAS: 556-67-2) and decamethylcyclopentasiloxane (CAS 541-02-6) are commonly employed in cosmetics and well known to a person skilled in the art. Often cyclomethicone (INCI nomenclature) is used in cosmetic compositions which refers to a mixture of several cyclic dimethyl polysiloxane compounds consisting essentially of octamethylcyclotetrasiloxane and decamethylcyclopentasiloxane.

The term ‘consisting essentially of octamethylcyclotetrasiloxane and decamethylcyclopentasiloxane’ as used herein means that the total amount of octamethylcyclotetrasiloxane and decamethylcyclopentasiloxane constitutes at least 90 wt.-%, preferably at least 95 wt.-%, most preferably at least 98 wt.-% of the mixture.

Preferred silicone oils in all embodiments according to the present invention are selected from the group consisting of cyclopentasiloxane, cyclotetrasiloxane, cyclohexasiloxane, octamethylcyclotetrasiloxane, decamethylcyclopentasiloxane, dimethicone, dimethiconol, dimethicone copolyol, phenyl trimethicone, methicone, simethicone, polysilicone-15 as well as mixtures thereof. More preferred silicone oils according to the present invention are octamethylcyclotetrasiloxane, decamethylcyclopentasiloxane, polysilicone-15 and dimethicone as well as mixtures thereof. Particularly advantageous in all embodiments of the present invention is the use of cyclomethicone consisting essentially of octamethylcyclotetrasiloxane and decamethylcyclopentasiloxane and polysilicone-15.

Polysilicone-15 (INCI nomenclature, CAS 207574-74-1) is also known as dimethicodiethylbenzal malonate and commercially available as PARSOL® SLX at DSM Nutritional Products Ltd.

Dimethicone (INCI nomenclature, CAS 63148-62-9) is also known as Polydimethylsiloxane.

In all embodiments of the present invention it is particularly preferred that the topical composition comprises only cyclomethicone consisting essentially of octamethylcyclotetrasiloxane and decamethylcyclopentasiloxane, only polysilicone-15 or only polysilicone-15 and dimethicone as silicone oil (in the absence of any further silicone oil).

The amount of the silicone oil(s) in the topical compositions according to the present invention can easily be adjusted by a person in the art. Preferably the total amount of silicone oil(s) is selected in the range of 0.1-50 wt.-%, more preferably in the range of 0.5 to 30 wt.-%, and most preferably in the range of 1 to 20 wt.-%.

In a particular advantageous embodiment, the silicone oil(s) constitute from 5-70 wt.-%, preferably from 5-50 wt.-%, most preferably from 10-40 wt.-% such as from 15-30 wt.-% of the total oil phase of the topical composition.

If polysilicone-15 or polysilicone-15 and dimethicone is/are used as sole silicone oil(s) in the topical compositions according to the present invention, the amount thereof is preferably selected in the range of 0.5-5 wt.-%, preferably in the range of 1-4 wt.-% and most preferably in the range of 2-3.5 wt.-% based on the total weight of the composition. In this case it is even more preferred to select the amount of polysilicone-15 or polysilicone-15 and dimethicone such that it constitutes only from 5-10 wt.-%, preferably from 5-7 wt.-% of the total oil phase.

The term oil phase refers to a phase consisting of any cosmetically acceptable oil as well as mixtures thereof, such oil being defined for the present purpose as any cosmetically acceptable material which is substantially insoluble in water and includes the silicone oil(s). As the oils can perform different functions in the topical composition, the specific choice is dependent on the purpose for which it is intended. Suitable oils may be volatile or non-volatile, or a mixture of both. Suitable volatile oils to be used in combination with the silicone oil(s) include, but are not limited to straight or branched chain hydrocarbons having from 8-20 carbon atoms, such as decane, dodecane, tridecane, tetradecane, and C₈₋₂₀ isoparaffins. Non-volatile oils include, but are not limited to, vegetable oils, such as coconut oil, jojoba oil, corn oil, sunflower oil, palm oil, soybean oil, carboxylic acid esters such as isostearyl neopentanoate, cetyl octanoate, cetyl ricinoleate, octyl palmitate, dioctyl malate, coco-dicaprylate/caprate, decyl isostearate, myristyl myristate, C₁₂₋₁₅ alkyl benzoate, animal oils such as lanolin and lanolin derivatives, tallow, mink oil or cholesterol; glyceryl esters, such as glyceryl stearate, glyceryl dioleate, glyceryl distearate, glyceryl linoleate, glyceryl myristate, and non-volatile hydrocarbons, such as isoparaffins, squalane, or petrolatum. A further suitable group of oils to be incorporated into the oil phase of the topical compositions according to the present invention include oil soluble sunscreen agents.

In all embodiments of the present invention, the amount of the porous polymethylmethacrylate bead is preferably selected in the range of 0.5 to 4 wt.-%, such as in particular in the range of 1.5 to 3.5 wt.-% based on the total weight of the composition.

In all embodiments of the present invention preferably the oil absorption capacity is selected in the range of 1.5-2.0 cc/g.

The oil absorption capacity refers to the weight of a specific oil absorbed by a material, determined by a specific method as outlined in the following. It includes the oil absorption capacity of the dry particles existing between the inherent voids within and on the surface of the particles. The oil absorption capacity as referred to in the present invention is determined at 23° C. by weighting 2 g of the respective beads into a 20 ml beaker glass. Then, liquid paraffin (Paraffinum Perliquidum PH.EUR. CAS 8042-47-5) is added. After addition of 4 to 5 drops of paraffin to the powder, mixing is performed using a spatula, and addition of paraffin is continued until conglomerates of oil and powder have formed. From this point, the paraffin is added one drop at a time and the mixture is then triturated with the spatula. The addition of oil is stopped when the loose and dry powder completely disappears and a highly viscous white to transparent homogeneous gel is obtained. The oil absorption capacity (cc/g) is then calculated by the volume of paraffin used (in cc) per g of the respective beads.

The particle size (in volume %) as given in the present invention is determined by a Coulter LS13320 or Malvern Mastersizer 2000 according to standard methods in the art.

The porous polymethylmethacrylate beads according to the present invention are preferably obtained by copolymerization of a monomer mixture consisting of methyl methacrylate and of ethylene glycol dimethacrylate in the presence of a porogen according to known methods in the art and as e.g. outlined in KR 2006036614 which is enclosed herein by reference.

The term ‘consisting of’ as used according to the present invention means that the total amount of monomers ideally sum up to 100 wt.-%. It is however not excluded that small amount of impurities or additives may be present such as e.g. in amounts of less than 5 wt.-%, preferably less than 3 wt.-% which are e.g. introduced via the respective raw materials.

The porogen is preferably selected from the group consisting of toluene, n-hexanone, methylisobutyl ketone and isoamyl alcohol.

Initiators for polymerizing the monomers to provide the porous polymethylmethacrylate beads of the invention are those which are normally suitable for free-radical polymerization of acrylate monomers and which are oil-soluble and have low solubility in water such as e.g. organic peroxides, organic peroxyesters and organic azo initiators. The initiator is generally used in an amount of about 0.01 to 1 wt.-% based on the total monomer content.

Optionally, a water soluble inhibitor can be added to inhibit polymerization in the water phase in order to prevent the formation of too much polymer by emulsion and/or solution polymerization in the water phase, which can result in bead agglomeration or emulsion type polymerization. Suitable inhibitors include those selected from thiosulfates, thiocyanates, water soluble hydroquinones and nitrites. When used, the water soluble inhibitor can generally be added in an amount of from about 0.01 to about 1 parts by weight based on 100 parts total monomer content.

Furthermore, a water soluble or water dispersible polymeric stabilizer is needed to stabilize the suspension and in order to obtain stable beads. The stabilizer is preferably a water soluble or water dispersible polymer such as e.g. polyvinylpyrrolidone, polyvinylmethylether, polyethyleneimine, polyvinylalcohol, gelatin, starch, (m)ethylcellulose, carboxymethylcellulose, hydroxyethylcellulose, hydroxypropyl ellulose, poly(meth)acrylic acid and their sodium salts, and the like. The stabilizer is preferably used in an amount of about 0.001 to 10 wt.-%, more preferable in an amount of about 0.01 to 1 wt-% based on the total monomer content.

The monomers, free-radical initiator, and any optional materials can be mixed together in the prescribed ratio to form a premix. The stabilizer can be combined with water and then with the premix to form an oil in water suspension. The resulting suspension typically comprises from about 10 to about 50 weight percent monomer premix and from about 90 to about 50 weight percent water phase. Bead-type suspension polymerization in accordance with the present invention is typically a thermally initiated polymerization and is preferably carried out with agitation for about 2 to about 16 hours at a temperature between about 40° C. and 90° C. After isolation of the beads according to standard methods such as filtration or centrifugation the beads are preferably washed e.g. with water and/or ethanol and subjected to an extended drying, preferably at about 40-100° C. and more preferably at about 80-100° C. in order to further reduce the residual monomer content to an amount of below 250 ppm such as in particular below 100 ppm. The drying can be performed by commonly known means to a person skilled in the art such as e.g. using a fluidized bed dryer or a conventional oven. The drying time can be easily adjusted by a person skilled in the art and is usually carried out over a period of 3 to 40 h such as about 8 to 20 h and in particular about 8 to 10 h.

In all embodiments of the present invention the porous polymethylmethacrylate beads are preferably prepared by suspension polymerisation of a monomer mixture consisting of 10-90 wt.-% methyl methacrylate and 10-90 wt.-% ethylene glycol dimethacrylate, with the proviso that the sum of monomers sums up to 100 wt.-%, in the presence of a porogen selected from toluene, n-hexanone, methylisobutyl ketone and isoamyl alcohol and a stabilizer selected from the group consisting of polyvinyl pyrrolidone, polyvinylmethylether, polyethyleneimine, poly(acrylicacid), polyvinylalcohol, vinyl acetate copolymer and ethyl cellulose.

Particularly suitable porous polymethylmethacrylate beads according to the present invention have a Dᵥ50 selected in the range of 9 to 12 μm and an oil absorption capacity selected in the range of 1.5-2.0 cc/g. Furthermore, it is preferred that the residual monomer content is less than 100 ppm, more preferably less than 50 ppm (determined by Gas Chromatography). It is furthermore advantageous if the beads exhibit as 10% aqueous dispersion in distilled water a pH in the range of 5.0 to 9.0. It is furthermore preferred if the porous polymethylmethacrylate beads have a water content of less than 1.5 wt.-% (determined by Karl Fischer titration).

Suitable porous polymethylmethacrylate beads according to the present invention having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g are e.g. commercially available as VALVANCE™ Touch 150 at DSM Nutritional Products Ltd Kaiseraugst.

The term “topical” is understood here to mean external application to keratinous substances, which are in particular the skin, scalp, eyelashes, eyebrows, nails, mucous membranes and hair.

Preferably, the topical compositions according to the present invention are applied to the skin.

As the compositions according to the invention are intended for topical application, they comprise a physiologically acceptable medium, that is to say a medium compatible with keratinous substances, such as the skin, mucous membranes, and keratinous fibers. In particular the physiologically acceptable medium is a cosmetically acceptable carrier.

The term cosmetically acceptable carrier refers to all carriers and/or excipients and/or diluents conventionally used in cosmetic compositions.

Preferred topical compositions according to the invention are skin care preparations or functional preparations.

Examples of skin care preparations are, in particular, light protective preparations (sunscreens), anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), skin lightening preparations, as well as BB and CC Creams.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment the topical compositions according to the invention are skin care preparations, such as (body) milks, lotions, foundations, creams, creamgels, serums, toners or gels.

The topical compositions according to the present invention may be in the form of a suspension or dispersion in solvents or fatty substances, or alternatively in the form of an emulsion or micro emulsion (in particular of oil-in-water (O/W) or water-in-oil (W/O) type, silicone-in-water (Si/W) or water-in-silicone (W/Si) type, PIT-emulsion, multiple emulsion (e.g. oil-in-water-in oil (O/W/O) or water-in-oil-in-water (W/O/W) type), pickering emulsion, hydrogel, alcoholic gel, lipogel, one- or multiphase solution or vesicular dispersion or other usual forms, which can also be applied by pens, as masks or as sprays.

In one embodiment, the topical compositions according to the present invention are advantageously in the form of an oil-in-water (O/W) emulsion comprising an oil phase dispersed in an aqueous phase in the presence of an O/W emulsifier. The preparation of such O/W emulsions is well known to a person skilled in the art and illustrated in the examples.

If the topical composition according to the invention is an O/W or Si/W emulsion, then it contains advantageously at least one O/W- or Si/W-emulsifier selected from the list of PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, Glyceryl Stearate Citrate, Glyceryl Stearate SE (self-emulsifying), stearic acid, salts of stearic acid, polyglyceryl-3-methylglycosedistearate. Further suitable emulsifiers are phosphate esters and the salts thereof such as cetyl phosphate (Amphisol® A), diethanolamine cetyl phosphate (Amphisol®DEA), potassium cetyl phosphate (Amphisol® K), sodiumcetearylsulfat, sodium glyceryl oleate phosphate, hydrogenated vegetable glycerides phosphate and mixtures thereof. Further suitable emulsifiers are sorbitan oleate, sorbitan sesquioleate, sorbitan isostearate, sorbitan trioleate, Cetearyl Glucoside, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C₁₀₋₃₀ alkyl acrylate crosspolymer, acrylates/steareth-20 methacrylate copolymer, PEG-22/dodecyl glycol copolymer, PEG-45/dodecyl glycol copolymer, and mixtures thereof.

The at least one 0/W and/or Si/W emulsifier is preferably used in an amount of 0.2 to 10 wt.-%, in particular in the range of 0.5 to 6 wt.-% such as more in particular in the range of 0.5 to 5 wt.-% such as most in particular in the range of 1 to 4 wt.-%, based on the total weight of the composition.

Particular suitable O/W emulsifiers according to the present invention encompass phosphate esters emulsifier of formula (II)

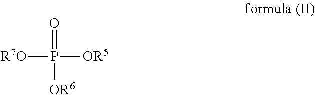

wherein R⁵, R⁶ and R⁷ may be hydrogen, an alkyl of from 1 to 22 carbons, preferably from 12 to 18 carbons; or an alkoxylated alkyl having 1 to 22 carbons, preferably from 12 to 18 carbons, and having 1 or more, preferably from 2 to 25, most preferably 2 to 12, moles ethylene oxide, with the provision that at least one of R⁵, R⁶ and R⁷ is an alkyl or alkoxylated alkyl as previously defined but having at least 6 alkyl carbons in said alkyl or alkoxylated alkyl group.

Monoesters in which R⁵ and R⁶ are hydrogen and R⁷ is selected from alkyl groups of 10 to 18 carbons and alkoxylated fatty alcohols of 10 to 18 carbons and 2 to 12 moles ethylene oxide are preferred. Among the preferred phosphate ester emulsifier are C₈₋₁₀ Alkyl Ethyl Phosphate, C₉₋₁₅ Alkyl Phosphate, Ceteareth-2 Phosphate, Ceteareth-5 Phosphate, Ceteth-8 Phosphate, Ceteth-10 Phosphate, Cetyl Phosphate, C6-10 Pareth-4 Phosphate, C₁₂₋₁₅ Pareth-2 Phosphate, C₁₂₋₁₅ Pareth-3 Phosphate, DEA-Ceteareth-2 Phosphate, DEA-Cetyl Phosphate, DEA-Oleth-3 Phosphate, Potassium cetyl phosphate, Deceth-4 Phosphate, Deceth-6 Phosphate and Trilaureth-4 Phosphate. A particular advantageous phosphate ester emulsifier according to the invention is potassium cetyl phosphate e.g. commercially available as Amphisol® K at DSM Nutritional Products Ltd Kaiseraugst.

Further suitable O/W emulsifiers are polyethyleneglycol (PEG) esters or diesters such as e.g. [INCI Names] PEG-100 Stearate, PEG-30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate.

Particularly preferred according to the present invention is PEG-100 Stearate sold under the tradename Arlacel™ 165 (INCI Glyceryl Stearate (and) PEG-100 Stearate) by Croda.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

In a particular embodiment, the invention relates to topical compositions in the form of O/W emulsions comprising an oil phase dispersed in an aqueous phase in the presence of an O/W emulsifier wherein the O/W emulsifier is selected from the group consisting of potassium cetyl phosphate, glyceryl stearate (and) PEG-100 Stearate, cetearyl olivate and sorbitan olivate as well as mixtures thereof.

Particular advantageous compositions according to the present invention are O/W emulsions comprising polysilicone-15 as sole silicone oil in an amount of 0.5-5 wt.-%, preferably in an amount of 1-4 wt.-% and most preferably in an amount of 2-3.5 wt.-% based on the total weight of the composition and potassium cetyl phosphate as emulsifier. In these compositions, the emulsifier is preferably used in an amount of 2 to 4 wt.-%, based on the total weight of the composition.

In another particular embodiment, the invention relates to topical compositions in the form of W/O emulsions comprising an aqueous phase dispersed in an oil phase in the presence of a W/O emulsifier. Preferred W/O emulsifiers are selected from the list of Polyglyceryl-3 Diisostearate, Polyglyceryl-2 Dipolyhydroxystearate, Polyglyceryl-4 Diisostearate/Polyhydroxystearate/Sebacate, Sorbitan Laurate, Sorbitan Trioleate and Methylglucose Diisostearate and low-ethoxylated compounds like PEG-7 hydrogenated castor oil. Particularly preferred are W/O emulsions wherein the W/O emulsifier is Polyglyceryl-3 Diisostearate, Polyglyceryl-2 Dipolyhydroxystearate and/or Polyglyceryl-4 Diisostearate/Polyhydroxystearate/Sebacate.

Thus, further particular advantageous compositions according to the present invention are W/O emulsions comprising polysilicone-15 or polysilicone-15 and dimethicone as sole silicone oil(s) in an amount of 0.5-5 wt.-%, preferably in an amount of 1-4 wt.-% and most preferably in an amount of 2-3.5 wt.-% based on the total weight of the composition and Polyglyceryl-4 diisostearate/polyhydroxystearate/sebacate as emulsifier. In these compositions, the emulsifier is preferably used in an amount of 2 to 4 wt.-%, based on the total weight of the composition.

In another preferred embodiment, the invention relates to W/Si emulsions comprising water dispersed in a silicone oil in the presence of a W/Si emulsifier. Preferably the W/Si emulsifier is selected from the list of cyclomethicone (and) PEG/PPG-18/18 dimethicone, lauryl PEG/PPG-18/18 methicone, cyclopentasiloxane (and) PEG-12 dimethicone crosspolymer, Cetyl PEG/PPG-10/1 Dimethicone, Dimethicone (and) Dimethicone PEG-10/15 Crosspolymer, Amodimethicone glycerocarbamate, bis-PEG/PPG-14/14 dimethicone (and) cyclopentasiloxane, Cyclopentasiloxane (and) PEG/PPG-20/15 Dimethicone, Cyclomethicone (and) PEG/PPG-20/15 Dimethicone, PEG/PPG-18/18 Dimethicone. Even more preferably, the silicone oil is selected from the group consisting of cyclopentasiloxane, cyclotetrasiloxane, cyclohexasiloxane, octamethylcyclotetrasiloxane as well as mixtures thereof. Even more preferred the silicone oil is selected from the group of cyclopentasiloxane, cyclotetrasiloxane, cyclohexasiloxane, octamethylcyclotetrasiloxane as well as mixtures thereof and the W/Si emulsifier is Cetyl PEG/PPG-10/1 Dimethicone (e.g. commercially available as Abil EM 90). Most advantageous compositions according to the present invention are W/Si emulsions comprising cyclomethicone consisting essentially of octamethylcyclotetrasiloxane and decamethylcyclopentasiloxane as sole silicone oil and Cetyl PEG/PPG-10/1 Dimethicone as emulsifier. In these compositions the amount of cyclomethicone is preferably selected in the range of 5 to 30-wt.-% such as in the range of 7.5 to 25 wt.-%. The Cetyl PEG/PPG-10/1 Dimethicone is preferably used in an amount selected in the range of 0.5 to 5 wt.-% such as more preferably in the range of 1.5 to 2.5 wt.-% based on the total weight of the W/Si emulsion.

The topical compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 5 wt.-%, such as most in particular in the range of 1 to 3 wt.-%, based on the total weight of the composition. Particular suitable co-surfactants are selected from the list of alkyl alcohols such as cetyl alcohol (Lorol C16, Lanette 16) cetearyl alcohol (Lanette O), stearyl alcohol (Lanette 18), behenyl alcohol (Lanette 22), glyceryl stearate, glyceryl myristate (Estol 3650), hydrogenated coco-glycerides (Lipocire Na10) as well as mixtures thereof.

The compositions in form of O/W, Si/W, W/O or W/Si emulsions according to the invention can be provided, for example, in all the formulation forms for emulsions, for example in the form of serum, milk, lotion or cream, and they are prepared according to the usual methods. The compositions which are subject-matters of the invention are intended for topical application and can in particular constitute a dermatological or cosmetic composition, for example intended for protecting human skin against the adverse effects of UV radiation (antiwrinkle, anti-ageing, moisturizing, anti-sun protection and the like).

According to an advantageous embodiment of the invention the compositions constitute cosmetic composition and are intended for topical application to the skin.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing, as well as agents to improve elasticity and skin barrier and/or further UV-filter substances and carriers and/or excipients or diluents conventionally used in topical compositions. If nothing else is stated, the excipients, additives, diluents, etc. mentioned in the following are suitable for topical compositions according to the present invention. The necessary amounts of the cosmetic and dermatological adjuvants and additives can, based on the desired product, easily be determined by the skilled person. The additional ingredients can either be added to the oil phase, the aqueous phase or separately as deemed appropriate. The mode of addition can easily be adapted by a person skilled in the art.

The cosmetically active ingredients useful herein can in some instances provide more than one benefit or operate via more than one mode of action.

The topical cosmetic compositions of the invention can also contain usual cosmetic adjuvants and additives, such as preservatives/antioxidants, fatty substances/oils, water, organic solvents, silicones, thickeners, softeners, emulsifiers, sunscreens, antifoaming agents, moisturizers, aesthetic components such as fragrances, surfactants, fillers, sequestering agents, anionic, cationic, nonionic or amphoteric polymers or mixtures thereof, propellants, acidifying or basifying agents, dyes, colorings/colorants, abrasives, absorbents, essential oils, skin sensates, astringents, antifoaming agents, pigments or nanopigments, e.g. those suited for providing a photoprotective effect by physically blocking out ultraviolet radiation, or any other ingredients usually formulated into cosmetic compositions. Such cosmetic ingredients commonly used in the skin care industry, which are suitable for use in the compositions of the present invention are e.g. described in the International Cosmetic Ingredient Dictionary & Handbook by Personal Care Product Council (http://www.personalcarecouncil.org/), accessible by the online INFO BASE (http://online.personalcarecouncil.org/jsp/Home.jsp), without being limited thereto.

In all embodiments of the present invention it is furthermore advantageous, if the topical compositions do not comprise any further silicone oils such as in particular linear polysiloxanes and most in particular dimethicone (polydimethylsiloxane).

The necessary amounts of the cosmetic and dermatological adjuvants and additives can—based on the desired product—easily be chosen by a skilled person in this field and will be illustrated in the examples, without being limited hereto.

Of course, one skilled in this art will take care to select the above mentioned optional additional compound or compounds and/or their amounts such that the advantageous properties intrinsically associated with the combination in accordance with the invention are not, or not substantially, detrimentally affected by the envisaged addition or additions.

The topical compositions according to the invention in general have a pH in the range of 3 to 10, preferably a pH in the range of 4 to 8 and most preferably a pH in the range of 4 to 7.5. The pH can easily be adjusted as desired with suitable acids such as e.g. citric acid or bases such as sodium hydroxide (e.g. as aqueous solution), Triethanolamine (TEA Care), Tromethamine (Trizma Base) and Aminomethyl Propanol (AMP-Ultra PC 2000) according to standard methods in the art.

The amount of the topical composition to be applied to the skin is not critical and can easily be adjusted by a person skilled in the art. Preferably the amount is selected in the range of 0.1−3 mg/cm² skin, such as preferably in the range of 0.1 to 2 mg/cm² skin and most preferably in the range of 0.5 to 2 wt.-%/cm².

The following examples are provided to further illustrate the compositions and effects of the present invention. These examples are illustrative only and are not intended to limit the scope of the invention in any way.

### EXAMPLE 1

### Sensory Evaluation

To 30 g of Nivea sun light feeling Gesichtsfluid (Beiersdorf, Art No. 85828, W/Si emulsion) Ingredients: Aqua, Cyclomethicone, Butylene Glycol Dicaprylate/Dicaprate, Glycerin, Octocrylene, Butyl Methoxydibenzoylmethane, Methylpropanediol, Sodium Phenylbenzimidazole Sulfonate, Bis-Ethylhexyloxyphenol Methoxyphenyl Triazine, Ethylhexyl Salicylate, Homosalate, C12-15 Alkyl Benzoate, Dicaprylyl Carbonate, Distarch Phosphate, Alcohol Denat., Titanium Dioxide, Tocopheryl Acetate, Cetyl PEG/PPG-10/1 Dimethicone, VP/Hexadecene Copolymer, Trimethoxycaprylylsilane, Trisodium EDTA, Phenoxyethanol, Methylparaben, Propylparaben, Linalool, Limonene, Butylphenyl Methylpropional, Benzyl Alcohol, Citronellol, Alpha-Isomethyl Ionone, Citral, Coumarin, Parfum) in a 100 ml beaker 3 wt.-% of VALVANCE™ Touch 150 (INCI: Methyl Methacrylate Crosspolymer; porous polymethylmethacrylate beads according to the invention from DSM Nutritional Products Ltd Kaiseraugst) was added under homogenization for 1 minute.

The sample prepared as outlined above was tested in a blind study with a trained sensorial panel consisting of 6 persons under the following conditions:

The evaluation takes part on the inner forearm; the panel leader applies 50 μL of the respective sample.

Evaluator spreads the product within a defined circle of 5 cm diameter using index or middle finger, circular motion, rate of 2 rotations/second. This is the so called rub-out phase. After the rub-out phase the silicone feel was assessed according to standardized parameters.

The intensities felt are quantified on a scale from 0 to 100 in comparison to training standards with known and defined sensory intensities.

The evaluation revealed a significantly improved silicone feel directly after application as well as after 20 min compared to the reference containing no porous polymethylmethacrylate beads according to the invention as outlined in table 1.

TABLE 1

| Silicone feel immediate | | Silicone feel 20 min | |
|---|---|---|---|
| Reference¹ | Sample¹ | Reference¹ | Sample² |
| 18 | 31 | 14 | 28 |

¹Reference = Nivea sun light feeling Gesichtsfluid
²Sample (acc. Invention) = Nivea sun light feeling Gesichtsfluid + 3 wt.-% of VALVANCE ™ Touch 150

### EXAMPLE 2

Two W/O emulsions A (sample according to the invention) and B (Reference) as outlined in table 2 were prepared. Afterwards a sensorial evaluation of these emulsions was carried out according to the method described in example 1.

TABLE 2
W/O emulsion

| | | | w/w | |
|---|---|---|---|---|
| | Tradename | INCI Name | A | B |
| A | PARSOL ® SLX | Polysilicone-15 | 3 | 3 |
| A | PARSOL ® 340 | Octocrylene | 10 | 10 |
| A | PARSOL ® 1789 | Butyl | 5 | 5 |
| | | methoxydibenzoylmethane | | |
| A | PARSOL ® EHS | Ethylhexyl salicylate | 5 | 5 |
| A | PARSOL ® TX50 AB | Titanium dioxide; | 6 | 6 |
| | | silica; dimethicone; | | |
| | | C₁₂₋₁₅ alkyl benzoate, | | |
| | | polyglyceryl-2 | | |
| | | dipolyhydroxystearate | | |
| A | Butylated Hydroxytoluene | BHT | 0.05 | 0.05 |
| A | Finsolv TN | C₁₂₋₁₅ alkyl benzoate | 2 | 2 |
| A | Myritol 318 | Caprylic/ | 4 | 4 |
| | | capric triglyceride | | |
| A | Myritol 331 | Cocoglycerides | 4 | 4 |
| A | Isolan GPS | Polyglyceryl- | 3 | 3 |
| | | 4 diisostearate/ | | |
| | | polyhydroxystearate/ | | |
| | | sebacate | | |
| A | Dermofeel ® BGC | Butylene glycol | 3 | 3 |
| | | dicaprylate/dicaprate | | |
| B | dl-alpha-Tocopheryl | Tocopheryl acetate | 0.5 | 0.5 |
| | Acetate | | | |
| C | Water dem. | Aqua | Ad 100 | |
| C | Magnesium Sulfate * 7 H₂O | Magnesium sulfate | 1 | 1 |
| C | 1,3-Butylenglycol | Butylene glycol | 10 | 10 |
| D | Euxyl PE 9010 | Phenoxyethanol, | 0.8 | 0.8 |
| | | ethylhexylglycerin | | |
| E | Water dem. | aqua | 2 | 5 |
| F | VALVANCE ™ | Methyl methacrylate | 3 | — |
| | Touch 150 | crosspolymer | | |

Procedure:

Heat part A (oil phase) and part B separately to 70° C. while stirring and add phase B to A. Then heat part C to 70° C. and add slowly to part AB while stirring and homogenizing the emulsion. Afterwards cool down to 35° C. and add part D. Under stirring add parts E and F (if present) and homogenize again.

The sensory evaluation revealed a significantly improved silicone feel directly after application as well as after 20 min compared to the reference containing no porous polymethylmethacrylate beads according to the invention as outlined in table 3.

TABLE 3

| Silicone feel immediate | | Silicone feel 20 min | |
|---|---|---|---|
| Sample B | Sample A | Sample B | Sample A |
| 3 | 12 | 8 | 28 |

### EXAMPLE 3

Two O/W emulsions C (sample according to the invention) and D (Reference) as outlined in table 4 were prepared. Afterwards a sensorial evaluation of these emulsions was carried out according to the method described in example 1.

TABLE 4
O/W emulsion

| | | | w/w | |
|---|---|---|---|---|
| | Tradename | INCI Name | C | D |
| A | PARSOL ®1789 | Butyl | 4 | 4 |
| | | methoxy- | | |
| | | dibenzoylmethane | | |
| A | PARSOL ® SLX | Polysilicone-15 | 2 | 2 |
| A | PARSOL ®340 | Octocrylene | 8 | 8 |
| A | PARSOL ®HMS | Homosalate | 4 | 4 |
| A | AMPHISOL ®K | Potassium | 3 | 3 |
| | | cetyl phosphate | | |
| A | Lanette O | Cetearyl alcohol | 2 | 2 |
| A | Butylated Hydroxytoluene | BHT | 0.05 | 0.05 |
| A | Euxyl PE 9010 | Phenoxyethanol, | 0.8 | 0.8 |
| | | ethylhexylglycerin | | |
| A | Dermofeel ®BGC | Butylene glycol | 8 | 8 |
| | | dicaprylate/dicaprate | | |
| A | Finsolv TN | C₁₂₋₁₅ alkyl benzoate | 4 | 4 |
| A | Antaron V-220 | VP/Eicosene copolymer | 0.5 | 0.5 |
| A | Cetiol CC | Dicaprylyl carbonate | 4 | 4 |
| A | Cetiol MM | Myristyl myristate | 1.5 | 1.5 |
| A | Keltrol CG-T | Xanthan gum | 0.3 | 0.3 |
| B | Glycerin 1,23 (86.5%) Ph. Eur. | Glycerin | 3 | 3 |
| B | Edeta BD | Disodium EDTA | 0.1 | 0.1 |
| B | Water dem. | Aqua | Ad 100 | |
| C | Triethanolamine Care | Triethanolamine | 0.3 | 0.3 |
| D | Water dem. | Aqua | 5 | 5 |
| D | Triethanolamine Care | Triethanolamine | 1.2 | 1.2 |
| D | PARSOL ®HS | Phenylbenzimidazol | 2 | 2 |
| | | sulfonic acid | | |
| E | VALVANCE ™ Touch 150 | Methyl methacrylate | 1 | — |
| | | crosspolymer | | |

Procedure:

Heat part A (oil phase) to 85° C. Heat part B to 80° C. Then add part C to part A and then part AC to part B during stirring. Homogenize at T about 70° C. Keep stirring till T<50° C. Add subsequently part D and E (if present) under mixing and homogenize again. Keep stirring till RT.

The sensory evaluation revealed a significantly improved silicone feel directly after application compared to the reference D as outlined in table 5.

TABLE 5
Silicone feel immediate

| Sample D | Sample C |
|---|---|
| 15 | 24 |

### Formulation Examples

### W/Si Emulsions

| Ingredients | Wt.-% | | |
|---|---|---|---|
| Phase A | | | |
| DOW CORNING ® | 7.0 | 7.0 | 7.0 |
| 5225C Formulation aid | | | |
| Abil ® EM-90 (Cetyl PEG/ | 2.0 | 2.0 | 2.0 |
| PPG-10/1 Dimethicone) | | | |
| Amphisol ® K (Potassium | — | 0.5 | 1.0 |
| Cetyl phosphate) | | | |
| Tegosoft ® TN (C12-15 | 8 | 8 | 8 |
| Alkyl Benzoate) | | | |
| DUB DIS (Dipentaerythrityl | 7.5 | 7.5 | 7.5 |
| hexacaprylate/hexacaprate) | | | |
| Cetiol ® CC (Dicaprylyl | 15.00 | 15.00 | 15.00 |
| Carbonate) | | | |
| DOW CORNING ® 344 (Blend | 12.0 | 12.0 | 12.0 |
| of Cyclotetrasiloxane & | | | |
| Cylcopentasiloxane) | | | |
| Phenonip | 0.8 | 0.8 | 0.8 |
| BHT | 0.1 | 0.1 | 0.1 |
| Abil ® Wax 9801 | 1.0 | 1.0 | 1.0 |
| Phase B | | | |
| Water dem. | Ad 100 | | |
| Sodium chloride | 0.5 | 0.5 | 0.5 |
| Glycerin 99.5% | 2. | 2.0 | 2.0 |
| Triethanolamine | 2.0 | 2.0 | 2.0 |
| EDTA | 0.1 | 0.1 | 0.1 |
| Phase C | | | |
| DOW CORNING ® 245 | 5.0 | 5.0 | 5.0 |
| (Cyclopentasiloxane) | | | |
| VALVANCE ™ Touch 150 | 5.0 | 4.0 | 2.0 |

Heat part A to 80° C. under stirring. Combine ingredients of part B until it is completely dissolved. Start to homogenize part A and add slowly part B. Homogenize for further 2 minutes at 17.500 rpm and let cool down under stirring. Disperse VALVANCE™ Touch 150 in part C. Add part C to the formulation at 40° C. and homogenize for 30 seconds at 17.500 rpm.

### W/O Lotions

| Ingredients | Wt.-% | | |
|---|---|---|---|
| Phase A | | | |
| Abil ® WE 09 (Polyglyceryl-4 | 5.0 | 5.0 | |
| Isostearate, Cetyl PEG/PPG-10/1 | | | |
| Dimethicone; Hexyl Laurate) | | | |
| Abil EM 90 (Cetyl PEG/PPG-10/1 | | | 2.5 |
| Dimethicone) | | | |
| Abil ® Wax 9801 (Cetyl Dimethicone) | 1.0 | 1.0 | |
| Abil ® Wax 9840 (Cetyl Dimethicone) | | | 0.25 |
| Tegosoft ® M (Isopropyl Myristate) | 4.0 | 4.0 | |
| Tegosoft ® OS (Ethylhexyl Stearate) | 4.0 | 4.0 | |
| Tegosoft ® CT (Caprylic/ | | | 13.0 |
| Capric Triglyceride) | | | |
| Cyclomethicone | 4.0 | | 2.0 |
| Dimethicone | | 4.0 | |
| Mineral Oil (30 mPas) | 5.0 | 5.0 | |
| Isohexadecane | | | 7.0 |
| Almond Oil | 2.0 | 2.0 | — |
| Argan oil | — | — | 1 |
| Hydrogenated Castor oil | 0.8 | 0.8 | 0.5 |
| Microcrystalline wax | 1.2 | 1.2 | 1.2 |
| Phase B | | | |
| Water dem. | Ad 100 | | |
| Sodium chloride | 0.50 | 0.80 | 0.60 |
| EDTA | 0.1 | 0.1 | 0.1 |
| Phase C | | | |
| VALVANCE ™ Touch 150 | 5.0 | 4.0 | 2.0 |
| Preservative, Parfum | q.s. | q.s. | q.s. |

Heat part A to 80° C. under stirring. Combine ingredients of part B until it is completely dissolved. Start to homogenize part A and add slowly part B. Homogenize for further 2 minutes at 17.500 rpm and let cool down under stirring. Add VALVANCE™ Touch 150 at 40° C. and homogenize for 30 seconds at 17.500 rpm.

### O/W Body Butter

| Ingredients | Wt.-% |
|---|---|
| Phase A | |
| Tego ® Care 165 (Glyceryl stearate; | 6.0 |
| PEG-100 Stearate) | |
| Tego ® Alkanol 1618 (Cetearyl Alcohol) | 1.5 |
| Tegosoft ® MM (Myristyl Myristate) | 1.0 |
| Tegosoft ® CR (Cetyl Ricinoleate) | 1.0 |
| Abil ® Wax 2240 | 1.0 |
| (Behenoxy Dimethicone) | |
| Cyclomethicone | 6.0 |
| Butyrospermum parkii (Shea butter) | 7.0 |
| Almond oil | 7.0 |
| Theobroma cacao (Cacaao butter) | 7.0 |
| Argan oil | 1.0 |
| Lanolin alcohol | 1.0 |
| Phase B | |
| Glycerin | 5.0 |
| Water dem. | Ad 100 |
| EDTA | 0.1 |
| Phase C | |
| VALVANCE ™ Touch 150 | 3.0 |
| Preservative, Parfum | q.s. |

Heat part A to 80° C. under stirring. Combine ingredients of part B until it is completely dissolved. Start to homogenize part A and add slowly part B. Homogenize for further 2 minutes at 17.500 rpm and let cool down under stirring. Add VALVANCE™ Touch 150 at 40° C. and homogenize for 30 seconds at 17.500 rpm.

1. A topical composition comprising at least one silicone oil and a porous cross-linked polymethylmethacrylate bead having a particle size Dᵥ0 of greater 0.3 μm, a Dᵥ100 of less than 35 μm, a Dᵥ50 selected in the range of 6 to 15 μm and an oil absorption capacity selected in the range of 1.2-2.5 cc/g in an amount selected in the range of 0.1 to 5 wt.-% based on the total weight of the composition, wherein the silicone oil(s) constitutes at least 5 wt.-% of the total oil phase of the topical composition.

2. The topical composition according to claim 1, wherein the amount of the porous polymethylmethacrylate bead is selected in the range of 0.5-4 wt.-% based on the total weight of the cosmetic composition.

3. The topical composition according to claim 1, wherein the particles size Dᵥ50 is selected in the range of 9 to 12 μm and the oil absorption capacity is selected in the range of 1.5-2.0 cc/g.

4. The topical composition according to claim 1, wherein the at least one silicone oil is selected from the group consisting of octamethylcyclotetrasiloxane, decamethylcyclopentasiloxane, dimethicone and polysilicone-15 as well as mixtures thereof.

5. The topical composition according to claim 4, wherein the at least one silicone oil is octamethylcyclotetrasiloxane and/or decamethylcyclopentasiloxane.

6. The topical composition according to claim 4, wherein the at least one silicone oil is polysilicone-15 or polysilicone-15 and dimethicone.

7. The topical composition according to claim 4, wherein the composition does not comprise any further silicone oil(s).

8. The topical composition according to claim 7, wherein the amount of polysilicone-15 is selected in the range of 0.5-5 wt.-%, preferably in the range of 1-4 wt.-% and most preferably in the range of 2-3.5 wt.-%, based on the total weight of the composition.

9. The topical composition according to claim 1, wherein the total amount of silicone oil(s) is selected in the range of 1-20 wt.-% based on the total weight of the composition.

10. The topical composition according to claim 1 wherein the porous polymethylmethacrylate bead is obtained by copolymerization of a monomer mixture consisting of methyl methacrylate and ethylene glycol dimethacrylate in the presence of a porogen.

11. The topical composition according to claim 1 wherein the topical composition is in the form of a W/Si emulsion comprising an aqueous phase dispersed in a silicone oil in the presence of a silicone emulsifier.

12. The topical composition according to claim 11, wherein the W/Si emulsifier is Cetyl PEG/PPG-10/1 Dimethicone.

13. The topical composition according to claim 1 wherein the silicone oil constitutes from 5-70 wt.-%, preferably from 5-50 wt.-%, most preferably from 10-40 wt.-%, such as from 15-30 wt.-% of the total oil phase of the topical composition.

14. Use of a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition to improve the silicone feel of a topical composition comprising a silicone oil on the skin, wherein the silicone oil constitutes at least 5 wt.-% of the total oil phase of the topical composition.

15. A method for improving the silicone feel of a topical composition comprising a silicone oil on skin, said method comprising the step of adding to the topical composition a porous polymethylmethacrylate bead having a particle size Dᵥ0 of greater 0.3 μm, a Dᵥ100 of less than 35 μm, a Dᵥ50 selected in the range of 6 to 15 μm and an oil absorption capacity in the range of 1.2-2.5 cc/g in an amount selected in the range of 0.1 to 5 wt.-% based on the total weight of the composition, applying the composition to the skin and appreciating the effect, wherein the silicone oil constitutes at least 5 wt.-% of the total oil phase of the topical composition.
